(19) United States
(12) Patent Application Publication
(10) Pub. No.: US 2002/0142456 A1
(43) Pub. Date: Oct. 3, 2002

(54) Canine OB protein compositions and methods

(75) Inventor: Natasha Hernday, Ventura, CA (US)

Correspondence Address: AMGEN INC., U.S. Patent Operations/JDE, Dept. 4300, M/S 27-4-A, One Amgen Center Drive, Thousand Oaks, CA, 91320-1799, US

(73) Assignee: Amgen Inc.

(21) Appl. No.: 09/789,306

(22) Filed: Feb. 20, 2001

Related U.S. Application Data
(63) Continuation of application No. 08/609,408, filed on Mar. 1, 1996, now abandoned.

(51) Int. Cl. C12P 21/08; C12N 5/10; C12N 5/06; C12N 5/02; C12N 5/00; A61K 38/00; C07K 17/00; C07K 16/00; C07K 7/00; C07K 5/00; C12N 15/74; C12N 15/70; C12N 15/63; C12N 15/09; C12N 15/00; C12N 1/00; C12P 21/06; C07H 21/04

(52) U.S. Cl. 435/350; 530/324; 536/23.5; 435/320.1; 435/325; 435/243; 435/69.1; 435/810; 530/387.7

(57) ABSTRACT

This invention provides for canine OB protein and associated nucleic acids, vectors, host cells, processes for production, related compositions, and methods for making and using thereof.

### FIELD OF THE INVENTION

The present invention relates to canine OB protein and related nucleic acids, vectors, host cells, methods of production, selective binding molecules, derivatives, pharmaceutical compositions, and diagnostic, therapeutic and cosmetic methods.

### BACKGROUND

Although the molecular basis for obesity is largely unknown, the identification of the “OB gene” and protein encoded (“OB protein”) has shed some light on mechanisms the body uses to regulate body fat deposition. Zhang et al., Nature 372: 425-432 (1994); see also, the Correction at Nature 374: 479 (1995). The OB protein is active in vivo in both ob/ob mutant mice (mice obese due to a defect in the production of the OB gene product) as well as in normal, wild type mice. The biological activity manifests itself in, among other things, weight loss. See generally, Barinaga, “Obese” Protein Slims Mice, Science 269: 475-476 (1995).

It is known, for instance, that in ob/ob mutant mice, administration of OB protein results in a decrease in serum insulin levels, and serum glucose levels. It is also known that administration of OB protein results in a decrease in body fat. This was observed in both ob/ob mutant mice, as well as non-obese normal mice. Pelleymounter et al., Science 269: 540-543 (1995); Halaas et al., Science 269: 543-546 (1995). See also, Campfield et al., Science 269: 546-549 (1995) (Peripheral and central administration of microgram doses of OB protein reduced food intake and body weight of ob/ob and diet-induced obese mice but not in db/db obese mice.) In none of these reports have toxicities been observed, even at the highest doses.

There is also an unmet need for treatment for obese animals, particularly pets. Obesity may cause cardiovascular problems incident to high blood lipid levels, artherial plaque, high cholesterol, and high blood pressure. Also, Type II diabetes is associated with obesity. All these conditions may be present in dogs, particularly dogs fed diets with inappropriate levels of protein or fat.

In addition, it may be desirable or profitable, particularly for show dogs, to breed dogs having desired amounts of fat.

It is therefore desirable to have a therapeutic or cosmetic composition which is safe and effective to administer to dogs for weight loss, fat loss, treatment of hyper- or dyslipidemias or diabetes.

### SUMMARY OF THE INVENTION

The present invention provides for canine OB protein and associated nucleic acids, vectors, host cells, processes for production, related compositions, and methods for making and using thereof.

### BRIEF DESCRIPTION OF THE DRAWINGS

FIG. 1: Presented is an alignment of the amino acid sequences for the present canine OB protein (top sequence, referred to as a pre-protein in reference to the presence of a leader sequence) and native human OB protein (Zhang et al., supra) (bottom, also referred to as a pre-protein). Using Program Manual for the Wisconsin Package, Version 8, September 1994, Genetics Computer Group, 575 Science Drive, Madison, Wis., USA the percent similarity is 90.476. Strictly using amino acid identity, the percent identity is 80.952.

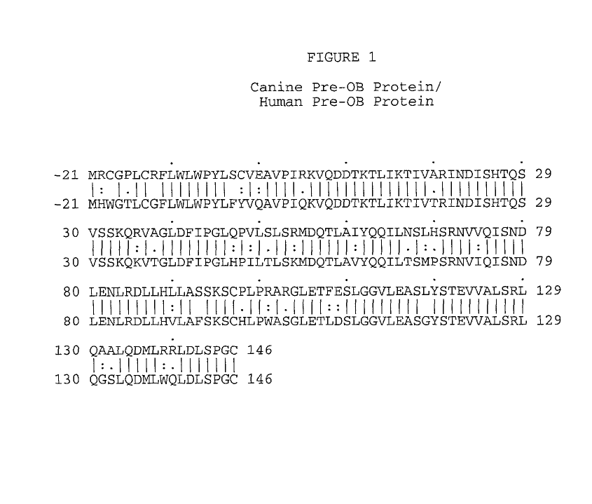

FIG. 2: Presented is an alignment of the amino acid sequences for the present canine OB protein (top sequence, again referred to as a pre-protein) and native murine OB protein (Zhang et al., supra) (bottom, also referred to as a pre-protein). Using the same analysis as in FIG. 1, above, the percent similarity is 86.905. Strictly using amino acid identity is 78.571.

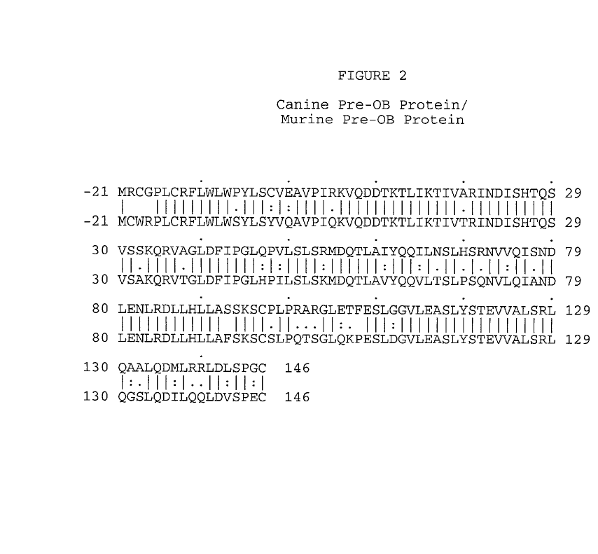

FIG. 3: Presented is an alignment of OB protein amino acid sequences of canine, human, murine and a consensus amino acid sequence. The consensus sequence listed is based on conserved amino acid regions. Amino acids at positions which differ between species are assigned an amino acid on the basis of which amino acid is most prevalent.

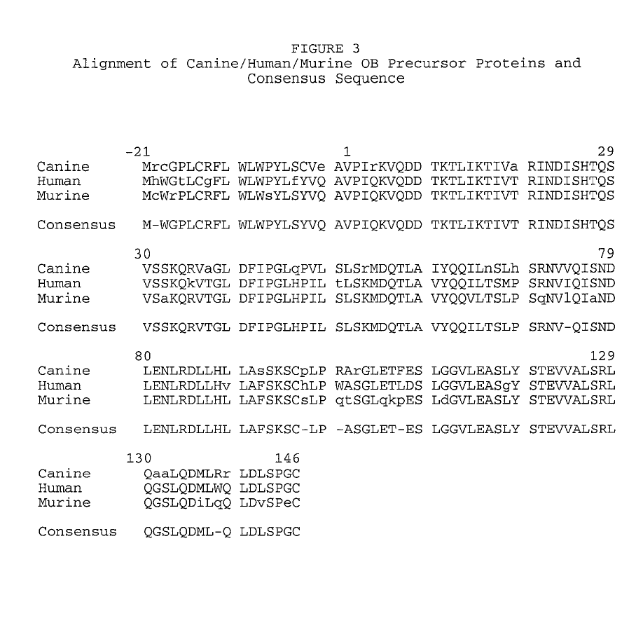

### DETAILED DESCRIPTION

The present invention provides for canine OB protein and associated nucleic acids, vectors, host cells, processes for production, related compositions, and methods for making and using thereof.

Protein Compositions

The full length canine OB protein is presented in Seq. ID. No. 1 (below). The leader sequence is (with respect to the numbering of Seq. ID No. 1) −21 to −1. The mature protein consists essentially of (with respect to the numbering of Seq. ID No. 1) 1 to 146. Also, the present canine OB protein may have present or absent the glutamine (“Q”) present at position +28 (with respect to the mature protein numbering of Seq.ID No. 1).One may choose to express the mature protein with an N-terminal methionyl residue incident to bacterial expression. Alternatively, one may choose to add other leader sequences for ease of expression.

Thus, the present canine OB protein may be selected from among the amino acid sequences (with respect to Seq. ID No. 1):

(a) −21 through 146;

(b) +1 through 146;

(c) +1 through 146 with an N-terminal methionine (referred to from time to time herein as “+1 through 146 met −1”); and

(d) an amino acid sequence of any of subparts (a), (b), or (c) above, lacking a glutaminyl residue at position 28.

As will be described more fully below, the present canine OB protein may optionally be formulated with a pharmaceutically acceptable diluent, adjuvant, or carrier.

Nucleic Acid Compositions and Methods

A nucleic acid encoding canine OB protein is presented in Seq. ID No. 2 (below). Novel nucleic acid sequences of the invention include sequences useful in securing expression in procaryotic or eucaryotic host cells of canine OB protein selected from among the amino acid sequences (with respect to Seq. ID No. 1):

(a) −21 through 146;

(b) +1 through 146;

(c) +1 through 146 with an N-terminal methionine; and,

(d) an amino acid sequence of any of subparts (a), (b), or (c) above, lacking a codon encoding a glutaminyl residue at position 28 (with respect to Seq. ID No. 1).

The nucleic acids may be purified and isolated, so that the desired coding region is useful to produce the present polypeptides, for example, or for diagnostic purposes, as described more fully below. DNA sequences of the invention specifically comprise: (a) the DNA of Seq. ID No. 2 (below); (b) the portion of the DNA sequence of Seq. ID No. 2 (below) encoding amino acids 1 through 146. Also comprehended are DNA sequences encoding allelic variant forms of canine OB protein, such as those forms encoding the amino acid sequence with the glutamine at position 28 absent, and manufactured DNA sequences encoding canine OB protein. Such manufactured sequences may readily be constructed according to the methods of Alton et al., PCT published application WO 83/04053.

Genomic DNA encoding the present canine OB protein may contain additional non-coding bases, or introns, and such genomic DNAs are obtainable by hybridizing all or part of the cDNA, illustrated in Seq. ID NO. 2, to a genomic DNA source, such as a canine genomic DNA library. Such genomic DNA will encode functional canine OB protein; however, use of the cDNAs may be more practicable in that, since only the coding region is involved, recombinant manipulation is facilitated.

The present nucleic acid sequences may include the incorporation of codons “preferred” for expression by selected nonmammalian hosts; the provision of sites for cleavage by restriction endonuclease enzymes; and the provision of additional initial, terminal or intermediate DNA sequences which facilitate construction of readily expressed vectors.

Also, one may prepare antisense nucleic acids against the present DNAs. Such antisense nucleic acids may be useful in modulating the effects of OB protein in vivo. For example, one may prepare an antisense nucleic acid which effectively disables the ability of a cell to produce canine OB protein.

DNA sequences of the invention are also suitable materials for use as labeled probes in isolating human genomic DNA encoding canine OB protein, as mentioned above, and related proteins as well as cDNA and genomic DNA sequences of other mammalian species. DNA sequences may also be useful in various alternative methods of protein synthesis (e.g., in insect cells) or in genetic therapy. DNA sequences of the invention are expected to be useful in developing transgenic mammalian species which may serve as eucaryotic “hosts” for production of and products in quantity. See, generally, Palmiter et al., Science 222: 809-814 (1983).

Vectors and Host Cells

According to another aspect of the present invention, the DNA sequences described herein which encode canine OB protein are valuable for the information which they provide concerning the amino acid sequence of the mammalian protein which have heretofore been unavailable. Put another way, DNA sequences provided by the invention are useful in generating new and useful viral and circular plasmid DNA vectors, new and useful transformed and transfected procaryotic and eucaryotic host cells (including bacterial and yeast cells and mammalian cells grown in culture), and new and useful methods for cultured growth of such host cells capable of expression of canine OB protein.

The DNA provided herein (or corresponding RNAs) may also be used for gene therapy for, example, treatment of conditions characterized by the insufficient expression of OB protein, such as obesity. Currently, vectors suitable for gene therapy (such as retroviral or adenoviral vectors modified for gene therapy purposes and of purity and pharmaceutical acceptability) may be administered for delivery into the lung, for example. Such vectors may incorporate nucleic acid encoding the present polypeptides for expression in a desired location. Gene therapy may involve a vector containing more than one gene for a desired protein. Alternatively, one may use no vector so as to facilitate relatively stable presence in the host. For example, homologous recombination may facilitate integration into a host genome. (This may be performed for production purposes as well, e.g., U.S. Pat. No. 5,272,071 and WO 91/09955.) The nucleic acid may be placed within a pharmaceutically acceptable carrier to facilitate cellular uptake, such as a lipid solution carrier (e.g., a charged lipid), a liposome, or polypeptide carrier (e.g., polylysine). A review article on gene therapy is Verma, Scientific American, November 1990, pages 68-84 which is herein incorporated by reference.

Also provided herein are methods for producing canine OD protein. Such methods for production include culturing, under suitable conditions, a host cell containing DNA encoding canine OB protein. Provided herein is an in vitro population of such host cells. Such host cell may be eukaryotic or prokayotic, and may contain a vector including the canine OB encoding DNA as provided herein. Alternatively, such canine OB protein DNA-containing host cell may have been modified via homologous recombination (see e.g., U.S. Pat. No. 5,272,071 and WO 91/09955 as mentioned supra) or other methods for altered expression of canine OB protein. One would then obtain via collection the desired canine OB protein. Optionally, one may further purify or process such canine OB protein, and prepare desired formulations.

Transgenic Animals

One may desire to produce dogs having altered canine OB levels. This may be accomplished by preparation of transgenic dogs. One way to prepare a transgenic dog is to genetically manipulate a dog embryo with a “transgene”, which typically includes a promoter and a desired DNA. In this case, the desired DNA may be selected from those enumerated above. The promoter may be for tissue specific expression, such as liver specific expression. One would then insert this transgene into the dog embryo. This may be accomplished by microinjection or by other means. One or more of such embryos is then implanted into a pseudo-pregnant foster mother dog. The embryo develops and the dog gives birth. One skilled in the art will recognize other means of preparing transgenic animals. Such animals may be transgenic for increased canine OB protein expression, thus making leaner animals, or transgenic to “knock-out” canine OB expression via, for example, using a defective canine OB DNA as a transgene. One or more of such animals may be prepared at a time (e.g., multiple embryo implants and multiple births); and such animals may be further bred. Thus, provided herein are transgenic dogs having altered canine OB protein expression. Such alteration may be in terms of amount (i.e., increased or decreased) or location of canine OB protein expression (i.e., tissue specific expression). Also provided herein are the progeny of such transgenic animals.

Such animals may be for pets, for show dogs, for breeding purposes, or for providing in vivo bioassays.

Derivatives, Pharmaceutical Compositions

The present protein may also be derivatized by the attachment of one or more chemical moieties to the protein moiety. The chemically modified derivatives may be further formulated for intraarterial, intraperitoneal, intramuscular, subcutaneous, intravenous, oral, nasal, pulmonary, topical or other routes of administration. Chemical modification of biologically active proteins has been found to provide additional advantages under certain circumstances, such as increasing the stability and circulation time of the therapeutic protein and decreasing immunogenicity. See U.S. Pat. No. 4,179,337, Davis et al., issued Dec. 18, 1979. For a review, see Abuchowski et al., in Enzymes as Drugs. (J. S. Holcerberg and J. Roberts, eds. pp. 367-383 (1981)). A review article describing protein modification and fusion proteins is Francis, Focus on Growth Factors 3: 4-10 (May 1992) (published by Mediscript, Mountview Court, Friern Barnet Lane, London N20, OLD, UK).

Chemical Moieties For Derivitization

The chemical moieties suitable for derivatization may be selected from among various water soluble polymers. The polymer selected should be water soluble so that the protein to which it is attached does not precipitate in an aqueous environment, such as a physiological environment. Preferably, for therapeutic use of the end-product preparation, the polymer will be pharmaceutically acceptable. One skilled in the art will be able to select the desired polymer based on such considerations as whether the polymer/protein conjugate will be used therapeutically, and if so, the desired dosage, circulation time, resistance to proteolysis, and other considerations. For the present proteins, the effectiveness of the derivatization may be ascertained by administering the protein or derivative, in the desired form (i.e., by osmotic pump, or, more preferably, by injection or infusion, or, further formulated for oral, pulmonary or nasal delivery, for example), and observing biological effects as described herein.

The water soluble polymer may be selected from the group consisting of, for example, polyethylene glycol, copolymers of ethylene glycol/propylene glycol, carboxymethylcellulose, dextran, polyvinyl alcohol, polyvinyl pyrolidone, polyl, 3-dioxolane, poly-1,3,6-trioxane, ethylene/maleic anhydride copolymer, polyaminoacids (either homopolymers or random or non-random copolymers), and dextran or poly(n-vinyl pyrolidone) polyethylene glycol, propylene glycol homopolymers, polypropylene oxide/ethylene oxide co-polymers, polyoxyethylated polyols, polystyrenemaleate and polyvinyl alcohol. Polyethylene glycol propionaldenhyde may have advantages in manufacturing due to its stability in water. The desired molecular weight of the polymer may be determined empirically, based on desired characteristics, as well as the degree of branching of the polymer.

Fusion proteins may be prepared by attaching polyaminoacids to the OB protein moiety. For example, the polyamino acid may be a carrier protein which serves to increase the circulation half life of the protein. For the present therapeutic or cosmetic purposes, such polyamino acid should be those which have do not create neutralizing antigenic response, or other adverse response. Such polyamino acid may be selected from the group consisting of serum album (such as canine serum albumin), an antibody or portion thereof (such as an antibody constant region, sometimes called “Fc”) or other polyamino acids. As indicated below, the location of attachment of the polyamino acid may be at the N-terminus of the OB protein moiety, or other place, and also may be connected by a chemical “linker” moiety, such as a polyamino acid linker, to the OB protein.

The number of polymer molecules so attached may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to protein (or peptide) molecules will vary, as will their concentrations in the reaction mixture. In general, the optimum ratio (in terms of efficiency of reaction in that there is no excess unreacted protein or polymer) will be determined by factors such as the desired degree of derivatization (e.g., mono, di-, tri-, etc.), the molecular weight of the polymer selected, whether the polymer is branched or unbranched, and the reaction conditions.

The chemical moieties should be attached to the protein with consideration of effects on functional or antigenic domains of the protein. There are a number of attachment methods available to those skilled in the art. E.g., EP 0 401 384 herein incorporated by reference (coupling PEG to G-CSF), see also Malik et al., Exp. Hematol. 20: 1028-1035 (1992) (reporting pegylation of GM-CSF using tresyl chloride). For example, a reactive group, such as, a free amino or carboxyl group may be used. The amino acid residues having a free amino group may include lysine residues and the N-terminal amino acid residue. Those having a free carboxyl group may include aspartic acid residues, glutamic acid residues, and the C-terminal amino acid residue. Sulfhydrl groups may also be used as a reactive group. Attachment at residues important for receptor binding should be avoided if receptor binding is desired.

One may specifically desire N-terminally chemically modified protein. Selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. For example, one may selectively N-terminally pegylate the protein by performing the reaction at a pH which allows one to take advantage of the pKₐ differences between the e-amino group of the lysine residues and that of the a-amino group of the N-terminal residue of the protein. By such selective derivatization, attachment of a water soluble polymer to a protein is controlled: the conjugation with the polymer takes place predominantly at the N-terminus of the protein and no significant modification of other reactive groups, such as the lysine side chain amino groups, occurs. Using reductive alkylation, the water soluble polymer may be of the type described above, and should have a single reactive aldehyde for coupling to the protein.

An N-terminally monopegylated derivative is preferred for ease in production of a therapeutic. N-terminal pegylation ensures a homogenous product as characterization of the product is simplified relative to di-, tri- or other multi pegylated products. The use of the above reductive alkylation process for preparation of an N-terminal product is preferred for ease in commercial manufacturing.

Pharmaceutical Compositions

In yet another aspect of the present invention, provided are methods of using pharmaceutical compositions of the proteins, and derivatives. Such pharmaceutical compositions may be for administration by injection, or for oral, pulmonary, nasal, transdermal or other forms of administration. In general, comprehended by the invention are pharmaceutical compositions comprising effective amounts of protein or derivative products of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used, and this may have the effect of promoting F sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations.

Contemplated for use herein are oral solid F dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). F Liposomal encapsulation may be used and the liposomes FH may be derivatized with various polymers (E.g., U.S. Pat. No. 5,013,556). A description of possible solid dosage forms for the therapeutic is given by Marshall, K. In: Modern Pharmaceutics Edited by G.S. Banker and C.T. Rhodes Chapter 10, 1979, herein incorporated by reference. In general, the formulation will include the protein (or analog or derivative), and inert ingredients which allow for protection against the stomach environment, and release of the biologically active material in the intestine.

Also specifically contemplated are oral dosage forms of the above derivatized proteins. Protein may be chemically modified so that oral delivery of the derivative is efficacious. Generally, the chemical modification contemplated is the attachment of at least one moiety to the protein (or peptide) molecule itself, where said moiety permits (a) inhibition of proteolysis; and (b) uptake into the blood stream from the stomach or intestine. Also desired is the increase in overall stability of the protein and increase in circulation time in the body. Examples of such moieties include: Polyethylene glycol, copolymers of ethylene glycol and propylene glycol, carboxymethyl cellulose, dextran, polyvinyl alcohol, polyvinyl pyrrolidone and polyproline. Abuchowski and Davis, Soluble Polymer-Enzyme Adducts. In: “Enzymes as Drugs”, Hocenberg and Roberts, eds., Wiley-Interscience, New York, N.Y., (1981), pp 367-383; Newmark, et al., J. Appl. Biochem. 4: 185-189 (1982). Other polymers that could be used are poly-1,3-dioxolane and poly-1,3,6-tioxocane.

For the protein (or derivative) the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a food or drink.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the protein (or derivative) are for instance the fatty acids oleic acid, linoleic acid and linolenic acid.

Controlled release formulation may be desirable. The drug could be incorporated into an inert matrix which permits release by either diffusion or leaching mechanisms i.e. gums. Slowly degenerating matrices may also be incorporated into the formulation. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some entric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Also contemplated herein is pulmonary delivery of the present protein, or derivative thereof. The protein (derivative) is delivered to the lungs of a mammal while inhaling and traverses across the lung epithelial lining to the blood stream. (Other reports of this include Adjei et al., Pharmaceutical Research 7: 565-569 (1990); Adjei et al., International Journal of Pharmaceutics 63: 135-144 (1990)(leuprolide acetate); Braquet et al., Journal of Cardiovascular Pharmacology 13(suppl. 5): s.143-146 (1989)(endothelin-1); Hubbard et al., Annals of Internal Medicine 3: 206-212 (1989)(a1-antitrypsin); Smith et al., J. Clin. Invest. 84: 1145-1146 (1989)(a-1-proteinase); Oswein et al., “Aerosolization of Proteins”, Proceedings of Symposium on Respiratory Drug Delivery II, Keystone, Colorado, March, 1990 (recombinant human growth hormone); Debs et al., The Journal of Immunology 140: 3482-3488 (1988)(interferon-g and tumor necrosis factor alpha) and Platz et al., U.S. Pat. No. 5,284,656 (granulocyte colony stimulating factor). Nasal delivery of the protein (or derivative) is also contemplated. Delivery via transport across other mucus membranes is also contemplated.

One skilled in the art will be able to ascertain effective dosages by administration and observing the desired therapeutic effect. Preferably, the formulation of the molecule will be such that between about 0.10 ag/kg/day and 1 g/kg/day will yield the desired therapeutic effect. The effective dosages may be determined using diagnostic tools over time. For example, a diagnostic for measuring the amount of OB protein in the blood (or plasma or serum) may first be used to determine endogenous levels of OB protein. Such diagnostic tool may be in the form of an antibody assay, such as an antibody sandwich assay. The amount of endogenous OB protein is quantified initially, and a baseline is determined. The therapeutic dosages are determined as the quantification of endogenous and exogenous OB protein (that is, protein, analog or derivative found within the body, either self-produced or administered) is continued over the course of therapy. The dosages may therefore vary over the course of therapy, with a relatively high dosage being used initially, until therapeutic benefit is seen, and lower dosages used to maintain the therapeutic benefits.

Selective Bindinq Molecules

A further embodiment of the invention is selective binding molecules, such as monoclonal antibodies selectively binding canine OB protein. The hybridoma technique described originally by Kohler and Milstein Eur. J. Immunol. 6, 511-519 (1976) has been widely applied to produce hybrid cell lines that secrete high levels of monoclonal antibodies against many specific antigens. Recombinant antibodies, (see Huse et al., Science 246: 1275 (1989)) may also be prepared. Such recombinant antibodies may be further modified, such as by modification of complementarity determining regions to increase or alter affinity, or alteration of constant regions to resemble canine constant regions of such antibodies. Such antibodies may be incorporated into a kit for diagnostic purposes, for example. A diagnostic kit may be employed to determine the location and/or amount of canine OB protein of an individual. Diagnostic kits, such as for a “sandwich” -type assay, may also be used to determine if an individual has receptors which bind canine OB protein, or those which, to varying degrees, have reduced binding capacity or ability. As stated infra, such antibodies may be prepared using immunogenic portions of a canine OB protein, particularly the immunogenic portions which are unique to canine OB protein.

Methods of Use

Therapeutic. Therapeutic uses include weight reduction, the treatment or prevention of diabetes, blood lipid reduction (and treatment of related conditions), increasing lean body mass and increasing insulin sensitivity. In addition, the present compositions may be used for manufacture of one or more medicaments for treatment or amelioration of the above conditions.

Weight Reduction. The present compositions and methods may be used for weight reduction. As has been demonstrated in murine models (see supra), administration of the present OB protein results in weight loss. The weight loss is primarily of adipose tissue, or fat. Such weight loss can be associated with the treatment of concomitant conditions, such as those below, and therefore constitute a therapeutic application. In addition, cosmetic uses are provided herein if weight loss is solely for improvement in appearance, such as for show dogs or dogs used for breeding.

Treatment of Diabetes. The present compositions and methods may be used in the prevention or treatment of Type II diabetes. As Type II diabetes can be correlated with obesity, use of the present invention to reduce weight can also alleviate or prevent the development of diabetes. Moreover, even in the absence of dosages sufficient to result in weight loss, the present compositions may be used to prevent or ameliorate diabetes.

Blood Lipid Reduction. The present compositions and methods may be used in the modulation of blood lipid levels. Ideally, in situations where solely reduction in blood lipid levels is desired, or where maintenance of reduction of blood lipid levels is desired, the dosage will be insufficient to result in weight loss. Thus, during an initial course of therapy of an obese animal, dosages may be administered whereby weight loss and concomitant blood lipid level lowering is achieved. Once sufficient weight loss is achieved, a dosage sufficient to prevent re-gaining weight, yet sufficient to maintain desired blood lipid levels may be administered. These dosages can be determined empirically, as the effects of OB protein are reversible. E.g., Campfield et al., Science 269: 546-549 (1995) at 547. Thus, if a dosage resulting in weight loss is observed when weight loss is not desired, one would administer a lower dose in order to achieve the desired blood lipid levels, yet maintain the desired weight.

Increasing Lean Mass or Insulin Sensitivity. Ideally, in situations where solely an increase in lean body mass is desired, the dosage will be insufficient to result in weight loss. Thus, during an initial course of therapy of an obese person, dosages may be administered whereby weight loss and concomitant fat F tissue decrease/lean mass increase is achieved. Once sufficient weight loss is achieved, a dosage sufficient to prevent re-gaining weight, yet sufficient to maintain desired lean mass increase (or, prevention of lean mass depletion) may be administered. These dosages can be determined empirically, as the effects of OB protein are reversible. E.g., Campfield et al., Science 269: 546-549 (1995) at 547. Thus, if a dosage resulting in weight loss is observed when weight loss is not desired, one would administer a lower dose in order to achieve the desired increase in lean tissue mass, yet maintain the desired weight. For increasing an individual's sensitivity to insulin, similar dosage considerations may be taken into account. Lean mass increase without weight loss may be achieved sufficient to decrease the amount of insulin (or, potentially, amylin or other potential diabetes treating drugs) an individual would be administered for the treatment of diabetes. For increasing overall strength, there may be similar dosage considerations. Lean mass increase with concomitant increase in overall strength may be achieved with doses insufficient to result in weight loss. Other benefits, such as an increase in red blood cells (and oxygenation in the blood) and a decrease in bone resorption or osteoporosis may also be achieved in the absence of weight loss.

Combination Therapies. The present compositions and methods may be used in conjunction with other therapies, such as altered diet and exercise. Other medicaments, such as those useful for the treatment of diabetes (e.g., insulin, and possibly amylin), cholesterol and blood pressure lowering medicaments (such as those which reduce blood lipid levels or other cardiovascular medicaments), activity increasing medicaments (e.g., amphetamines), and appetite suppressants. Such administration may be simultaneous or may be in seriatim. In addition, the present methods may be used in conjunction with surgical procedures, such as cosmetic surgeries designed to alter the overall appearance of a body (e.g., liposuction or laser surgeries designed to reduce body mass, or implant surgeries designed to increase the appearance of body mass). The health benefits of cardiac surgeries, such as bypass surgeries or other surgeries designed to relieve a deleterious condition caused by blockage of blood vessels by fatty deposits, such as arterial plaque, may be increased with concomitant use of the present compositions and methods. Methods to eliminate gall stones, such as ultrasonic or laser methods, may also be used either prior to, during or after a course of the present therapeutic methods. Furthermore, the present methods may be used as an adjunct to surgeries or therapies for broken bones, damaged muscle, or other therapies which would be improved by an increase in lean tissue mass.

Diagnostic

The present canine OB protein or nucleic acids may be used for diagnostic purposes. For instance, RNAs or DNAs may be used to characterize or detect defects in an individuals canine OB DNA gene or gene product. For example, an obese individual may have a defective canine OB gene. The present DNAs may be used to hybridize with the nucleic acid from an individual to detect such defects, such as via PCR techniques. Canine OB protein may be used to characterize an individual's OB protein for its ability to bind to OB receptor, or for other biological activity. For example, one may prepare an assay for the ability of oD protein to alter lipid metabolism by preparing a population of lipid containing cells expressing the OB receptor, and contacting OB protein with such population of cells. Modulation of lipid content, characteristics of lipid or other characteristics may be monitored. For diagnostic purposes, the present protein or nucleic acids may be associated with a detectable labels substance such as a radioactive isotope, a fluorescent or chemiluminescent chemical, or other label available to one skilled in the art. Such nucleic acids may be used for tissue distribution assays (for example, to detect the distribution of canine OB mRNA transcripts in different tissue types) or for other assays to determine the location of OB receptor.

### AMINO ACID AND DNA SEQUENCES

The following are sequence listings for the present canine OB protein and related DNA.

For the amino acid sequence (Seq. ID No. 1), the first amino acid of the mature protein is at position +1 and is a valine (“V”) and is indicated in bold print. One may add a methionyl residue at position −1, with the elimination of the leader sequence, for ease in expression in bacterial systems. Alternatively, one may have other N-terminal modifications, such as recognition sites for agents which cleave amino acids, for ease in production of protein without the N-terminal methionyl residue. For example, one may include an enzymatic recognition site at the N-terminus for ease in production (abbreviated as “1-146 met −1”). In addition, one may prepare a version with the glutamine at position 28 absent.

As stated above, one may alter the DNA (Seq. ID. No. 2) yet not alter the amino acid sequence which is encoded. Such alterations may be, for example, to include restriction sites for ease in replication or vector insertion, or to include codons preferred for expression in certain systems, such as bacterial expression or eukaryotic expression.

Canine OB Protein (Seq. ID No. 1)

| | |
|---|---|
| −21 | MRCGPLCRFL WLWPYLSCVE AVPIRKVQDD TKTLIKTIVA |
| | RINDISHTQS |
| 30 | VSSKQRVAGL DFIPGLQPVL SLSRMDQTLA IYQQILNSLH |
| | SRNVVQISND |
| 80 | LENLRDLLHL LASSKSCPLP RARGLETFES LGGVLEASLY |
| | STEVVALSRL |
| 130 | QAALQDMLRR LDLSPGC* |

Canine OB Protein DNA (Seq. ID No. 2)

| | |
|---|---|
| 1 | ATGCGTTGTG GACCTCTGTG CCGATTCCTG TGGCTTTGGC |
| | CCTATCTGTC |
| 51 | CTGTGTTGAA GCTGTGCCAA TCCGAAAAGT CCAGGATGAC |
| | ACCAAAACCC |
| 101 | TCATCAAGAC GATTGTCGCC AGGATCAATG ACATTTCACA |
| | CACGCAGTCT |
| 151 | GTCTCCTCCA AACAGAGGGT CGCTGGTCTG GACTTCATTC |
| | CTGGGCTCCA |
| 201 | ACCAGTCCTG AGTTTGTCCA GGATGGACCA GACGTTGGCC |
| | ATCTACCAAC |
| 251 | AGATCCTCAA CAGTCTGCAT TCCAGAAATG TGGTCCAAAT |
| | ATCTAATGAC |
| 301 | CTGGAGAACC TCCGGGACCT TCTCCACCTG CTGGCCTCCT |
| | CCAAGAGCTG |
| 351 | CCCCTTGCCC CGGGCCAGGG GCCTGGAGAC CTTTGAGAGC |
| | CTGGGCGGCG |
| 401 | TCCTGGAAGC CTCACTCTAC TCCACAGAGG TGGTGGCTCT |
| | GAGCAGACTG |
| 451 | CAGGCGGCCC TCCAGGACAT GCTTCGGCGG CTGGACCTCA |
| | GCCCTGGGTG |
| 501 | CTGA |

While the present invention has been described in terms of preferred embodiments, it is understood that variations and modifications will occur to those skilled in the art. Therefore, it is intended that the appended claims cover all such equivalent variations which come within the scope of the invention as claimed.

```
# SEQUENCE LIS
#TING
(1) GENERAL INFORMATION:
      (iii) NUMBER OF SEQUENCES: 5
(2) INFORMATION FOR SEQ ID NO: 1:
          (i) SEQUENCE CHARACTERISTICS:
                    (A) LENGTH: 167 amino
#acids
                    (B) TYPE: amino acid
                    (C) STRANDEDNESS: single
                    (D) TOPOLOGY: linear
        (ii) MOLECULE TYPE: protein
        (ix) FEATURE:
                    (A) NAME/KEY: Leader Se
#quence
                    (B) LOCATION: -21 to
#-1
        (xi) SEQUENCE DESCRIPTION: SEQ ID NO:
#1:
Met Arg Cys Gly Pro Leu Cys Arg Phe Leu Tr
#p Leu Trp Pro Tyr Le
        -20
# -15
# -10
Ser Cys Val Glu Ala Val Pro Ile Arg Lys Va
#l Gln Asp Asp Thr Ly
-5
#1 5
# 10
Thr Leu Ile Lys Thr Ile Val Ala Arg Ile As
#n Asp Ile Ser His Th
                        15
# 20
# 25
Gln Ser Val Ser Ser Lys Gln Arg Val Ala Gl
#y Leu Asp Phe Ile Pr
                30
# 35
# 40
Gly Leu Gln Pro Val Leu Ser Leu Ser Arg Me
#t Asp Gln Thr Leu Al
        45
# 50
# 55
Ile Tyr Gln Gln Ile Leu Asn Ser Leu His Se
#r Arg Asn Val Val Gl
60
#65
#70
#75
Ile Ser Asn Asp Leu Glu Asn Leu Arg Asp Le
#u Leu His Leu Leu Al
                                80
# 85
# 90
Ser Ser Lys Ser Cys Pro Leu Pro Arg Ala Ar
#g Gly Leu Glu Thr Ph
                        95
# 100
# 105
Glu Ser Leu Gly Gly Val Leu Glu Ala Ser Le
#u Tyr Ser Thr Glu Va
                110
# 115
# 120
Val Ala Leu Ser Arg Leu Gln Ala Ala Leu Gl
#n Asp Met Leu Arg Ar
        125
# 130
# 135
Leu Asp Leu Ser Pro Gly Cys
140 1
#45
(2) INFORMATION FOR SEQ ID NO: 2:
          (i) SEQUENCE CHARACTERISTICS:
                    (A) LENGTH: 504 base
#pairs
                    (B) TYPE: nucleic acid
                    (C) STRANDEDNESS: single
                    (D) TOPOLOGY: linear
        (ii) MOLECULE TYPE: cDNA
        (xi) SEQUENCE DESCRIPTION: SEQ ID NO:
#2:
ATGCGTTGTG GACCTCTGTG CCGATTCCTG TGGCTTTGGC CCTATCTGTC CT
#GTGTTGAA 60
GCTGTGCCAA TCCGAAAAGT CCAGGATGAC ACCAAAACCC TCATCAAGAC GA
#TTGTCGC 120
AGGATCAATG ACATTTCACA CACGCAGTCT GTCTCCTCCA AACAGAGGGT CG
#CTGGTCT 180
GACTTCATTC CTGGGCTCCA ACCAGTCCTG AGTTTGTCCA GGATGGACCA GA
#CGTTGGC 240
ATCTACCAAC AGATCCTCAA CAGTCTGCAT TCCAGAAATG TGGTCCAAAT AT
#CTAATGA 300
CTGGAGAACC TCCGGGACCT TCTCCACCTG CTGGCCTCCT CCAAGAGCTG CC
#CCTTGCC 360
CGGGCCAGGG GCCTGGAGAC CTTTGAGAGC CTGGGCGGCG TCCTGGAAGC CT
#CACTCTA 420
TCCACAGAGG TGGTGGCTCT GAGCAGACTG CAGGCGGCCC TCCAGGACAT GC
#TTCGGCG 480
CTGGACCTCA GCCCTGGGTG CTGA
#
# 504
(2) INFORMATION FOR SEQ ID NO: 3:
          (i) SEQUENCE CHARACTERISTICS:
                    (A) LENGTH: 167 amino
#acids
                    (B) TYPE: amino acid
                    (C) STRANDEDNESS: single
                    (D) TOPOLOGY: linear
        (ii) MOLECULE TYPE: protein
        (ix) FEATURE:
                    (A) NAME/KEY: Leader Se
#quence
                    (B) LOCATION: -21 to
#-1
        (xi) SEQUENCE DESCRIPTION: SEQ ID NO:
#3:
Met His Trp Gly Thr Leu Cys Gly Phe Leu Tr
#p Leu Trp Pro Tyr Le
        -20
# -15
# -10
Phe Tyr Val Gln Ala Val Pro Ile Gln Lys Va
#l Gln Asp Asp Thr Ly
-5
#1 5
# 10
Thr Leu Ile Lys Thr Ile Val Thr Arg Ile As
#n Asp Ile Ser His Th
                        15
# 20
# 25
Gln Ser Val Ser Ser Lys Gln Lys Val Thr Gl
#y Leu Asp Phe Ile Pr
                30
# 35
# 40
Gly Leu His Pro Ile Leu Thr Leu Ser Lys Me
#t Asp Gln Thr Leu Al
        45
# 50
# 55
Val Tyr Gln Gln Ile Leu Thr Ser Met Pro Se
#r Arg Asn Val Ile Gl
60
#65
#70
#75
Ile Ser Asn Asp Leu Glu Asn Leu Arg Asp Le
#u Leu His Val Leu Al
                                80
# 85
# 90
Phe Ser Lys Ser Cys His Leu Pro Trp Ala Se
#r Gly Leu Glu Thr Le
                        95
# 100
# 105
Asp Ser Leu Gly Gly Val Leu Glu Ala Ser Gl
#y Tyr Ser Thr Glu Va
                110
# 115
# 120
Val Ala Leu Ser Arg Leu Gln Gly Ser Leu Gl
#n Asp Met Leu Trp Gl
        125
# 130
# 135
Leu Asp Leu Ser Pro Gly Cys
140 1
#45
(2) INFORMATION FOR SEQ ID NO: 4:
          (i) SEQUENCE CHARACTERISTICS:
                    (A) LENGTH: 167 amino
#acids
                    (B) TYPE: amino acid
                    (C) STRANDEDNESS: single
                    (D) TOPOLOGY: linear
        (ii) MOLECULE TYPE: protein
        (ix) FEATURE:
                    (A) NAME/KEY: Leader Se
#quence
                    (B) LOCATION: -21 to
#-1
        (xi) SEQUENCE DESCRIPTION: SEQ ID NO:
#4:
Met Cys Trp Arg Pro Leu Cys Arg Phe Leu Tr
#p Leu Trp Ser Tyr Le
        -20
# -15
# -10
Ser Tyr Val Gln Ala Val Pro Ile Gln Lys Va
#l Gln Asp Asp Thr Ly
-5
#1 5
# 10
Thr Leu Ile Lys Thr Ile Val Thr Arg Ile As
#n Asp Ile Ser His Th
                        15
# 20
# 25
Gln Ser Val Ser Ala Lys Gln Arg Val Thr Gl
#y Leu Asp Phe Ile Pr
                30
# 35
# 40
Gly Leu His Pro Ile Leu Ser Leu Ser Lys Me
#t Asp Gln Thr Leu Al
        45
# 50
# 55
Val Tyr Gln Gln Val Leu Thr Ser Leu Pro Se
#r Gln Asn Val Leu Gl
60
#65
#70
#75
Ile Ala Asn Asp Leu Glu Asn Leu Arg Asp Le
#u Leu His Leu Leu Al
                                80
# 85
# 90
Phe Ser Lys Ser Cys Ser Leu Pro Gln Thr Se
#r Gly Leu Gln Lys Pr
                        95
# 100
# 105
Glu Ser Leu Asp Gly Val Leu Glu Ala Ser Le
#u Tyr Ser Thr Glu Va
                110
# 115
# 120
Val Ala Leu Ser Arg Leu Gln Gly Ser Leu Gl
#n Asp Ile Leu Gln Gl
        125
# 130
# 135
Leu Asp Val Ser Pro Glu Cys
140 1
#45
(2) INFORMATION FOR SEQ ID NO: 5:
          (i) SEQUENCE CHARACTERISTICS:
                    (A) LENGTH: 167 amino
#acids
                    (B) TYPE: amino acid
                    (C) STRANDEDNESS: single
                    (D) TOPOLOGY: linear
        (ii) MOLECULE TYPE: protein
        (ix) FEATURE:
                    (A) NAME/KEY: Leader Se
#quence
                    (B) LOCATION: -21 to
#-1
        (xi) SEQUENCE DESCRIPTION: SEQ ID NO:
#5:
Met Xaa Trp Gly Pro Leu Cys Arg Phe Leu Tr
#p Leu Trp Pro Tyr Le
        -20
# -15
# -10
Ser Tyr Val Gln Ala Val Pro Ile Gln Lys Va
#l Gln Asp Asp Thr Ly
-5
#1 5
# 10
Thr Leu Ile Lys Thr Ile Val Thr Arg Ile As
#n Asp Ile Ser His Th
                        15
# 20
# 25
Gln Ser Val Ser Ser Lys Gln Arg Val Thr Gl
#y Leu Asp Phe Ile Pr
                30
# 35
# 40
Gly Leu His Pro Ile Leu Ser Leu Ser Lys Me
#t Asp Gln Thr Leu Al
        45
# 50
# 55
Val Tyr Gln Gln Ile Leu Thr Ser Leu Pro Se
#r Arg Asn Val Xaa Gl
60
#65
#70
#75
Ile Ser Asn Asp Leu Glu Asn Leu Arg Asp Le
#u Leu His Leu Leu Al
                                80
# 85
# 90
Phe Ser Lys Ser Cys Xaa Leu Pro Xaa Ala Se
#r Gly Leu Glu Thr Xa
                        95
# 100
# 105
Glu Ser Leu Gly Gly Val Leu Glu Ala Ser Le
#u Tyr Ser Thr Glu Va
                110
# 115
# 120
Val Ala Leu Ser Arg Leu Gln Gly Ser Leu Gl
#n Asp Met Leu Xaa Gl
        125
# 130
# 135
Leu Asp Leu Ser Pro Gly Cys
140 1
#45
```

1. A canine OB protein selected from among amino acids (according to the numbering of Seq. ID. No. 1):
(a) −21 through 146;
(b) +1 through 146;
(c) +1 through 146 met −1; and,
(d) a canine OB protein of any of subparts (a), (b), or (c) above lacking a glutaminyl residue at position 28;
optionally in a pharmaceutically acceptable diluent, adjuvant or carrier.

2. A purified and isolated DNA encoding a canine OB protein selected from among amino acids (according to the numbering of Seq. ID. No. 1):
(a) −21 through 146;
(b) +1 through 146; and, (c) +1 through 146 met −1.
(d) a canine OB protein of any of subparts (a), (b), or (c) above lacking a glutaminyl residue at position 28.

3. A DNA of claim 2 which is a cDNA.

4. A DNA of claim 2 which is a genomic DNA.

5. A DNA of claim 2 which is selected from among (according to Seq. ID No. 2):
(a) that portion of Seq. ID No. 2 which encodes canine OB protein −22 through +146 (according to Seq. ID No. 1);
(b) that portion of Seq. ID No. 2 which encodes canine OB protein +1 through +146 (according to Seq. ID No. 1);
(c) the DNA of subpart (b) also encoding an methionyl residue at position −1; and, (d) the DNA of any of subparts (a), (b) or (c) above lacking a codon for glutamine at position 28 with respect to Seq. ID No. 1.

6. A vector containing a DNA according to any of claims 2, 3, 4, or 5.

7. A eukaryotic or prokaryotic host cell containing a DNA according to any of claims 2, 3, 4, or 5; or containing a vector containing a DNA according to any of claims 2, 3, 4, or 5.

8. A process for producing canine OB protein comprised of culturing, under suitable conditions, a population of prokaryotic or eukaryotic host cells containing a DNA according to any of claims 2, 3, 4, or a vector containing a DNA according to any of claims 2, 3, 4, or 5, and obtaining the canine OB protein product produced.

9. A selective binding molecule which selectively binds canine OB protein.

10. A monoclonal antibody which selectively binds canine OB protein.

11. A kit containing a selective binding molecule which selectively binds canine OB protein.

12. A method of treating a dog comprised of administering a therapeutically effective amount of canine OB protein according to claim 1.

13. A method of claim 12 wherein said dog is treated for obesity, type II diabetes, elevated blood lipid levels, or to increase lean body mass.

14. A transgenic dog having altered canine OB protein expression.
